(19) United States
(12) Patent Application Publication
(10) Pub. No.: US 2025/0185961 A1
(43) Pub. Date: Jun. 12, 2025

(54) IMPROVED SYSTEM FOR COLLECTION OF BLOOD

(71) Applicant: SOL-MILLENNIUM SWISS R&D CENTER SA, LUGANO (CH)

(72) Inventors: Marco BELLUZZI, COMO (CO) (IT); Dario DE ZOLT, FAGNANO OLONA (VA) (IT); Matteo LAGANA', LONGONE AL SEGRINO (CO) (IT); Andrea MASPERO, VENEGONO INFERIORE (VA) (IT)

(73) Assignee: SOL-MILLENNIUM SWISS R&D CENTER SA, LUGANO (CH)

(21) Appl. No.: 18/846,027

(22) PCT Filed: Mar. 23, 2023
(86) PCT No.: PCT/IB2023/052871

§ 371 Date: Sep. 11, 2024

(30) Foreign Application Priority Data
Apr. 6, 2022 (IT) 102022000006836

(51) Int. Cl. A61B 5/15

(52) U.S. Cl. CPC A61B 5/150267; A61B 5/15003

(57) ABSTRACT

A system for collection of blood in a phial has a cylindrical unit which can receive this phial or bottle at least partially, the cylindrical unit having an inner cavity and having a first, open end and a second end, inside the cavity of this unit there being removably inserted an element which can contain an empty test tube. The test tube is integral with this element, such as to constitute therewith a single component present in the cylindrical unit, which component is blocked in this unit for taking of the first blood, but is separable from the test tube only after this first blood has been collected.

The subject of the present invention is a system for collection of blood in a phial according to the preamble of the main claim.

The system which is the subject of the present invention is a system which is used to permit collection of blood in conventional phials or bottles in order to carry out blood culture thereof, or an examination of a microbiological type which is used to identify any microorganisms present in the blood itself. This examination is carried out on blood which is usually collected in a phial with a predetermined volume (in general for adults between 8 and 10 ml).

In order to collect the blood correctly and safely, in general a needle inserted in a venous or arterial vessel is used; the blood is taken and sent to a system which permits collection thereof in the phial which is connected by means of a conventional tube to said needle. In particular, the free end of the tube (which is not connected to the needle) is coupled in a known manner to a unit for retention of the phial;

this unit for retention of the phial (which is part of the system for collection of the blood) is a cylindrical unit with an open first end through which an end of the known phial is inserted. In a known manner, the phial comprises an opening which is closed by a perforable membrane, said membrane being perforated by a needle which is present inside a cavity of this cylindrical unit at a second, closed end of said cylindrical unit. Through said tube (which is connected to the needle introduced into the blood vessel) the blood taken from a patient is thus collected in the phial.

The cylindrical unit or unit for retention of the phial comprises a body having at least two portions with different diameters or transverse cross-sections: a first portion close to the first end of this cylindrical unit is larger than the second portion ending with the second end of this unit; the first portion of said cylindrical unit can contain partially the phial, the end of which that is provided with an opening is placed in the second portion of said cylindrical unit (where it is penetrated by the needle present there).

The collection of blood is an operation which must be carried out with the greatest care in order to prevent contamination of the blood itself by the organisms which are naturally present on the patient's skin. If these organisms are not perfectly removed by the disinfection procedure which precedes insertion of the needle into the blood vessel, they can reach the blood vessel itself, drawn by the needle during the insertion, and thus be aspirated into the blood culture phial, giving rise to “false positives”.

The need to reduce the “false positives” arises in order to avoid unnecessary antibiotic treatments, which can weaken the patient further, or constitute a treatment which is ineffective against pathologies which are really in progress, as well as increase the costs of the therapy. In particular, it is advisable and good laboratory practice to discard from the subsequent examination a quantity of blood which is taken first from the blood vessel of the patient, in order to avoid false positives in the subsequent blood culture.

In order to achieve the above, it is known to insert in the cylindrical unit or unit for retention of the phial an element which can contain a test tube for collection under vacuum, which receives a first flow of blood, and will not be used in the blood culture, which blood is subsequently discarded in a manner which is known, safe, and in conformity with the regulations in force. However, this methodology is not compulsory, i.e. the instruments which permit taking of the blood do not oblige an operator carrying out this operation to collect and discard the first flow of blood (or “first blood”) taken, and retain only the flow taken subsequently and put into the phial to be used for the subsequent examination.

The present invention relates to a system which obliges the operator carrying out the collection to discard the “first blood” which has been taken after the needle has been inserted into the blood vessel, and has been collected in a test tube.

Various solutions are known which describe a system comprising a unit which can be connected to a phial for collection of the blood, and has internally an element which can support an empty test tube.

For example, EP1708955 describes a system for supporting a phial for the transfer of fluid, comprising, inside the unit which can cooperate with the phial, an adapter which can be inserted in said unit through a first, open end thereof. This adapter has a base with a central hole and a cylindrical tube which extends from this opening, and the diameter of which is slightly smaller than the diameter of the first, open end of the unit which can cooperate with the phial. This tube can receive an empty test tube in order to collect the “first blood”.

Around the tube there project elements which can cooperate with the wall of said first end, such as to block the adapter in said unit, and prevent undesired removal thereof. On the other hand, when the adapter is removed, said unit can accept various containers with different diameters, such as, for example, phials with different dimensions.

EP1622506 describes an apparatus to transfer a fluid comprising a unit which can cooperate with a plurality of containers for a bodily fluid (blood) with various diameters and dimensions, and in which there is placed a sleeve which substantially fills an inner cavity of said unit, and is provided with a plurality of projecting wings, which centre this sleeve in this cavity. The wings cooperate with the inner surface of the cavity of said unit before the insertion of the sleeve in the cavity ends, such as to guide an end part of a cylindrical portion of the sleeve towards a needle inside said cavity. The sleeve makes it possible to associate with said unit a plurality of containers (vials, etc.), and at the same time to retain them stably in said unit. In addition, this prevents a healthcare worker from being able to reach the needle inside said unit, with a potential threat to safety.

Other solutions of the type described in the aforementioned patent texts are described in U.S. Pat. Nos. 9,192,327 and 4,320,769.

The objective of the present invention is to provide a system for collection of blood in a phial of the aforementioned type, which is improved in comparison with the known solutions.

In particular, the objective of the present invention is to provide a system of the aforementioned type in which it is possible to collect and discard the “first blood”, i.e. the blood which is taken from a blood vessel of the patient immediately after insertion into the patient of the needle (connected by means of a tube to said unit), which first blood can be the source of false positives when the blood is examined (such as, for example, in blood culture).

Another objective is to provide a system of the aforementioned type which obliges the healthcare worker carrying out the collection to collect and discard this first blood.

A further objective is to provide a system of the aforementioned type which is simple for this healthcare worker to use, and which, after the first blood has been collected, makes it possible to take the required quantity of blood, and collect it in the desired phial, for example for blood culture.

Another objective is to provide a system of the aforementioned type which is simple and economical to produce.

These objectives and others, which will become apparent to persons skilled in the art, are achieved by a system for collection of blood in a phial according to the appended claims.

For better understanding of the present invention, the following drawings are appended by way of non-limiting example, in which:

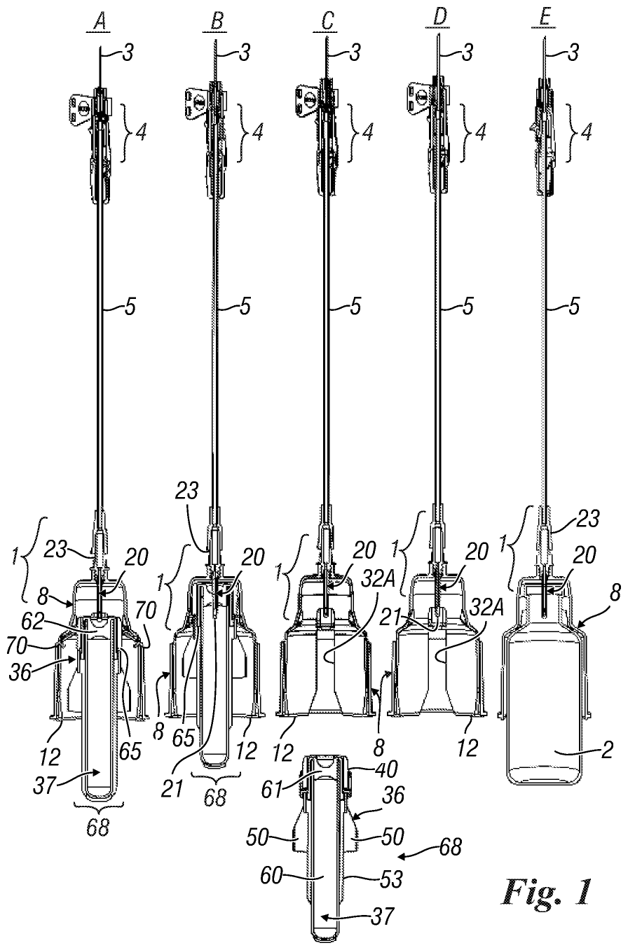

FIG. 1 shows a view in transverse cross-section of the system according to the invention used in combination with a microperfuser needle for taking blood, said system being shown in the various stages, indicated as A, B, C, D, E, of its use;

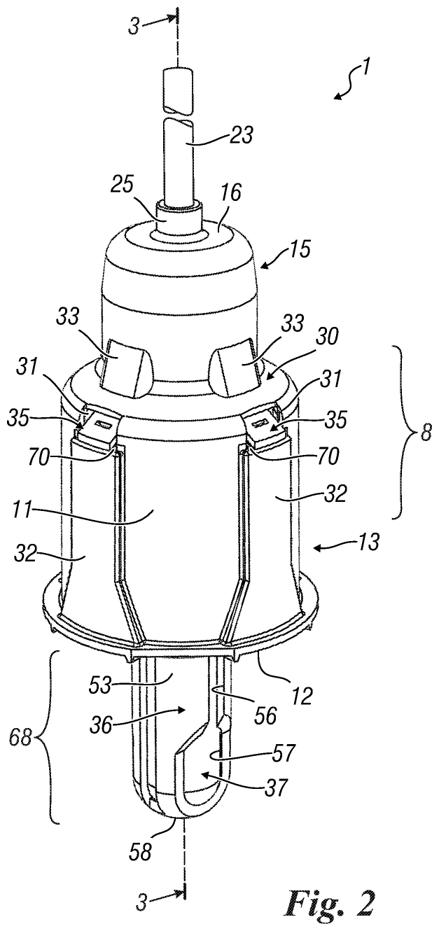

FIG. 2 shows a view in perspective of the system of FIG. 1;

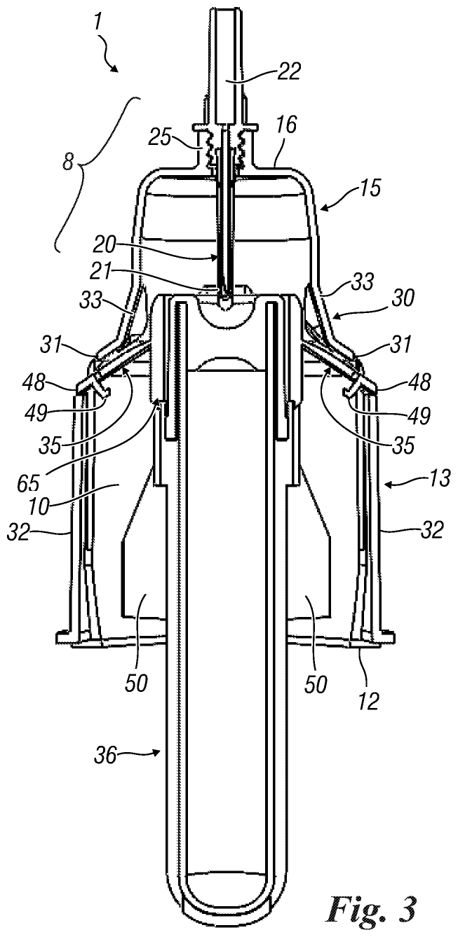

FIG. 3 shows a view in cross-section along the line 3-3 of FIG. 2;

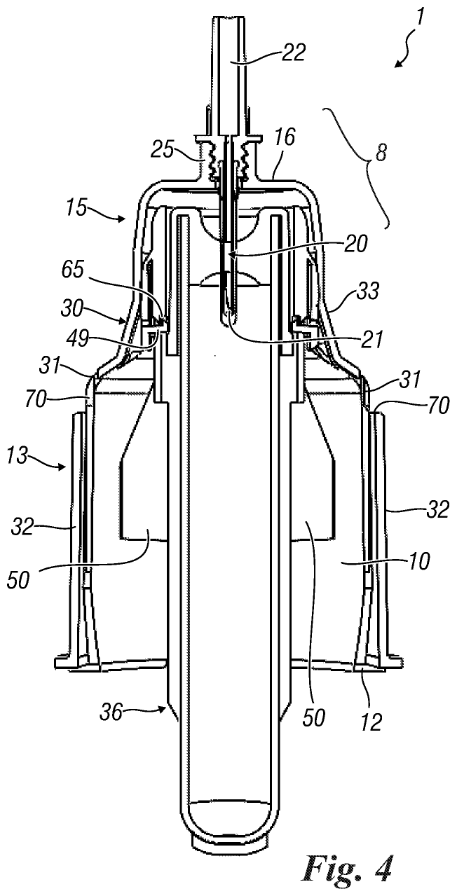

FIG. 4 shows a view in cross-section similar to that of FIG. 3, but with the system in a different stage of use;

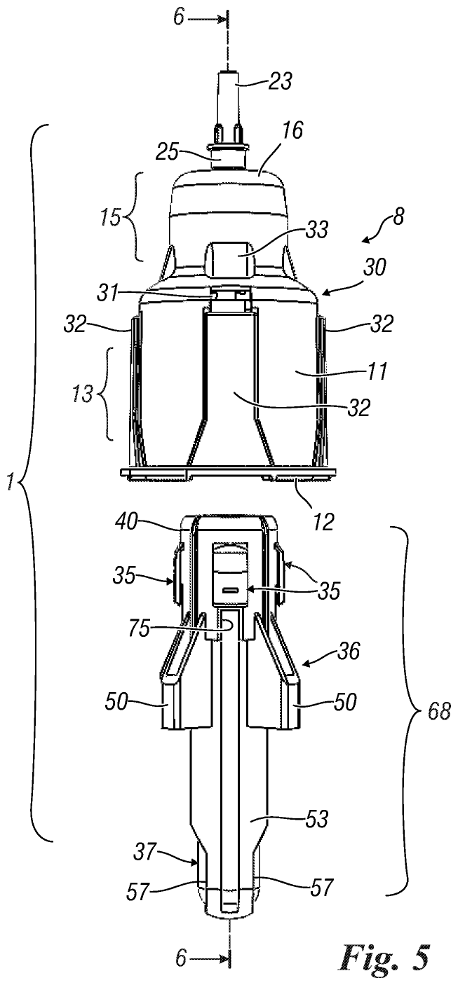

FIG. 5 shows a view similar to that of FIG. 3 in a further stage of use of the system;

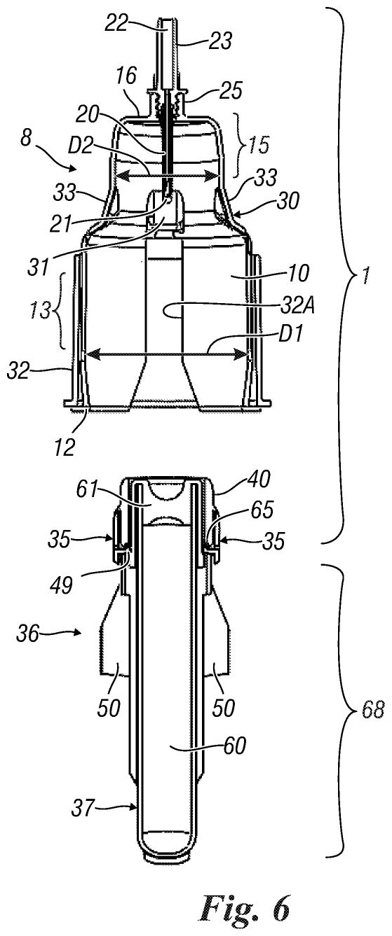

FIG. 6 shows a view in cross-section along the line 6-6 of FIG. 5;

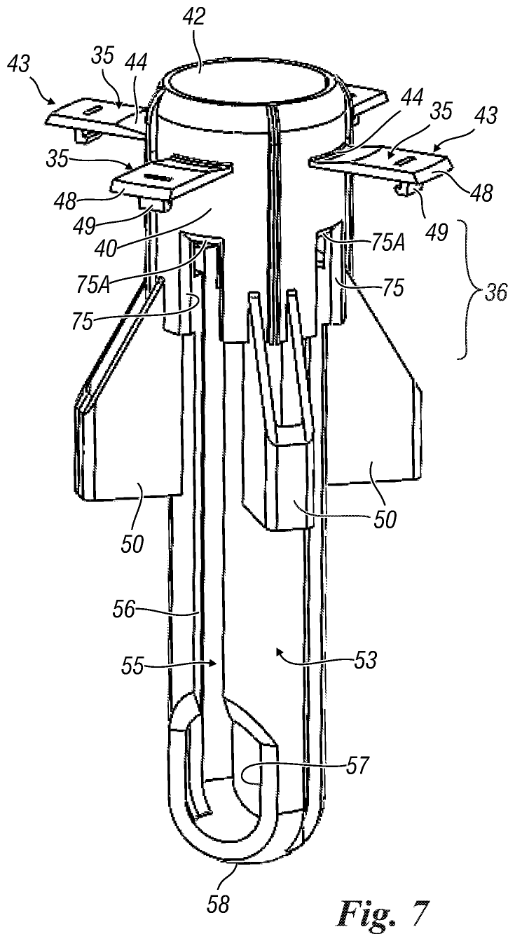

FIG. 7 shows a view in perspective of part of the system according to the invention;

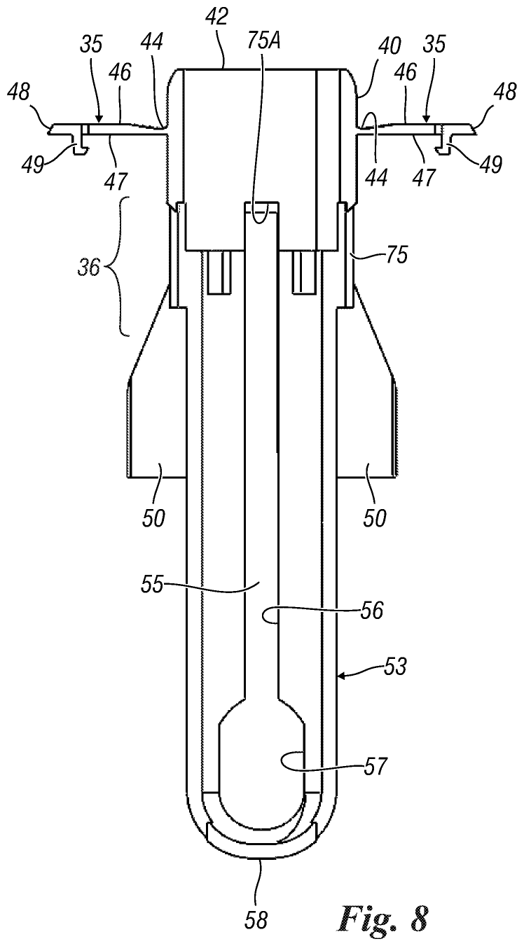

FIG. 8 shows a view in longitudinal cross-section of part of FIG. 7.

With reference to the aforementioned figures, a system according to the invention is generally indicated as 1, and can permit the collection of blood in a known bottle or phial 2 in order to subject the blood to an examination such as blood culture. The blood is for example taken through a needle 3 of a conventional microperfuser needle 4 connected by means of a 30 known tube 5 to said system 1.

The system 1 according to the invention comprises a cylindrical unit 8 which can retain and support the phial 2 during the transfer and collection of this blood in the phial 2 itself; this unit for retention of the phial has a substantially cylindrical form, and is hollow internally at 10, said cavity 10 being delimited by a wall 11. The unit for retention of the phial (hereinafter known as a “cylindrical unit”) 8 comprises a first portion 13 with a diameter or cross-section D1, and having a first, open end 12 of the cylindrical unit; a second portion 15, with a cross-section or diameter D2 smaller than D1 (see FIG. 6) is connected to the first portion 13, and ends in a second end 16 of the cylindrical unit 8. Screwed onto this second end there is a needle 20 with a tip 21 which faces towards the first, open end 12 of the cylindrical unit, and penetrates into the second portion 15 of the cylindrical unit. When associated with the cylindrical unit 8, the phial occupies the first 13 and the second 15 portions of said unit, in a known manner.

The needle 20 is secured on the cylindrical unit 8, and is thus part of said cylindrical unit. This needle can be secured on said cylindrical unit 8 also in a manner different from that described above.

The needle 20 is connected to a hole 22 of a duct 23 of a connection element (for example of the Luer type) 25; said duct projects from the cylindrical unit 8, and the tube 5 can be attached to it in any known manner.

The wall 11 of the cavity 10 and thus the cylindrical unit 8 has a (third) portion 30 which connects the first and the second portions, in which there are provided openings or windows 31, and, corresponding to these, strengthening protuberances 32 provided on the surface of the first portion 13. These protuberances define internally “channels” 32A which help in the assembly of the system 1. This third portion defines a sort of inclined connection part on the cylindrical unit 8, and also has inclined areas 33 at the openings or windows 31 which project outside the cylindrical unit 8, and connect the portion 15 to the portion 30.

The windows or openings 31 can cooperate with wings 35 of an element or trap 36 (which is also part of the system 1, like the unit for retention of the phial or cylindrical unit 8), which element can contain in a blocked manner an empty test tube 37, for example, or similar containers. The assembly of the element or trap 36 with the cylindrical unit 8 is facilitated by the channels 32A which guide the wings 35 towards the windows or openings 31. When the wings 35 of the element or trap 36 are inserted in said openings, they block the element or trap 36 (with the respective test tube 37) inside the cylindrical unit 8. The wings 35 can however be separated from said openings or windows 31 by means of a one-way movement (i.e. they can be released in a one-way manner), as will be described hereinafter.

More particularly, the element or trap 36 has a first part 40, which defines a first end of said element or trap, and can be positioned in the vicinity of the second end 16 of the cylindrical unit 8, inside the second portion 15 thereof. This first part 40 is open at 42, and supports the wings 35 which project outside it.

Each wing has a body 43 which is connected, by means of a portion with a reduced cross-section 44 acting as a resilient hinge, to the part 40; each wing has opposite flat surfaces 46, 47 with a free end 48, and, preferably, an element in the form of a hook 49 projecting from the surface 47, and underneath it in the figures.

From the part 40 there also extend laterally fins 50 which can facilitate the grasping of the element or trap 36 by a user or operator; on the other hand, a second part or body 53 extends below (in the figures) said first part 40, and defines a container portion for the empty test tube 37 (of a type which in itself is known and does not form part of the present invention). This container body or portion has an inner cavity 55 which can contain and retain by interference the test tube, and opposite lateral slots 56; in the vicinity of a closed end 58 thereof, there are present large openings 57 which can make it possible to extract the test tube 37 from the part or body 53. This end 58 is curved like the empty test tube 37, such as to follow its shape in contact. Preferably, the second part 53 of the element or trap 36 is made of flexible material, such as polyethylene.

When they are coupled, the element or trap 36 and the empty test tube 37 define a component 68 which acts as a single body inside the cylindrical unit 8 from the point of view of movement relative to this unit. In other words, when the test tube 37 is inside the element or trap 36, they define a single component for the purposes of insertion into, and extraction from, the cylindrical unit 8, with the test tube of said component being able to receive the “first blood” which emerges from the blood vessel (vein or artery) when the needle 3 is inserted into it.

The empty test tube 37 is of a type which in itself is known, and comprises a container part 60 which is open on one side where there is a stopper 61 which is provided with a perforable membrane 62, and has a transverse dimension or diameter larger than that of the container part. A step 65 is thus present between the stopper and said container part.

With the element or trap 36 inserted inside the cylindrical unit 8, the wings 35 (as previously stated) are positioned inside the openings or windows 31 of the cylindrical unit, and are supported with their free end 48 on the edge 70 of these windows. In this situation, the element or trap 36 is blocked inside the cylindrical unit 8, and the needle 20 is close to the membrane 62 of the stopper 61 (see FIG. 1, phase A, and FIG. 3) of the test tube 37.

When the cylindrical unit 8 is connected, the tube 5 of the microperfuser needle 4 and the needle 3 thereof is inserted into the patient's blood vessel, and by acting on the element or trap 36, the component 68 is moved towards the second end 16 of the cylindrical unit 8 (FIG. 1, phase B), such that the needle 20, secured on this cylindrical unit, can penetrate in the empty test tube 37, by passing through the perforable membrane 62, and the first blood taken from the patient can be collected in this test tube.

It should be noted that by means of this (one-way) movement of the component 68 towards the end 16, the wings 35 slide on the inclined areas 33 of the cylindrical unit 8, they emerge from the windows 31, and are folded back towards the empty test tube, towards openings 75 provided in the first part 40 or first end of the element or trap 36. By this means, by continuing the one-way movement of the component 68 towards said end 16, the wings 35 fold back completely onto the empty test tube 37, and, if they are present, the ends in the form of the hook 49 of the wings hook (snap) onto the upper edge 75A of the corresponding openings 75. This is shown in phase B of FIG. 1 and in FIG. 4.

It should be noted that, in any case, this test tube (inserted by means of interference in the element or trap 36) can advantageously be extracted from this element or trap. The configuration with a test tube which can be extracted is advantageous if it is wished to test the first blood taken, or if it is wished to dispose differently of the test tube with the blood to be discarded and the element or trap.

At this point, by acting on the element or trap 36, it is possible to extract the component 68 from the cylindrical unit 8 (phase C of FIG. 1 and FIGS. 5 and 6), since this element or trap 36 is free from any connection with the cylindrical unit 8, and it is possible to insert inside the unit 8 (which is empty, phase D of FIG. 1) the phial 2 (phase E of FIG. 1), such as to be able to collect the blood as desired for subsequent analysis. This phial is of a known type, and is not part of the system 1 according to the invention.

The above description represents a correct method of use of a system for collection of blood according to the invention, which method maintains the intactness of the parts, i.e. the cylindrical unit 8 and the element or trap 36, and avoids any problems for the healthcare worker who is taking blood.

It will be appreciated that, since the cylindrical unit 8 and the element or trap 36 are made of plastic material (as is usual), the separation between them can take place incorrectly, including by forcing the extraction of this element or trap 36 from the cylindrical unit 8 in the direction opposite that described thanks to the folding back of the wings 35.

This second method of extraction is not correct, and it will be appreciated that it is not recommended.

Preferably, the component 68 is arranged with the empty test tube 37 inserted (in a manner which is non-removable or extractable) in the second part 53 of the element or trap 36, before the coupling with the cylindrical unit 8, and before use of the system 1 for taking of blood; by this means, a healthcare worker (or other employee) carrying out said collection must necessarily eliminate from the examination the “first blood” taken when the needle 3 is introduced into the blood vessel. There is therefore no risk that the blood subsequently examined (for example by means of blood culture) will contain microorganisms which are naturally present on the skin (perforated by the needle 3) which may not have been removed completely during the usual disinfection procedure before insertion of the needle 3 into the blood vessel. These microorganisms may have been carried inside the blood vessel drawing the insertion of the needle, and have been immediately aspirated during the taking of the blood, thus contaminating the sample obtained and directed towards the system 1.

The solution is simple, convenient for the operator, and easy to use.

According to a possible variant, the cylindrical unit 8 can also be without windows 31, and the wings 35 can be produced such as to block themselves inside the cavity 10 of this cylindrical unit, for example by means of their surface parts which “harpoon” and “bite” the wall 11 which delimits this cavity.

It should be noted that, in all cases, when the element or trap 36 is inserted in the cylindrical unit 8, and before it is displaced towards the end 16 thereof, this element or trap and the entire component 68 can not be extracted easily from this cylindrical unit by pulling them to the exterior of the cavity 10; this is because of the high resistance of the wings 35, which, with their end 48, are supported on the edge 70 of the windows 31. This resistance makes the user perceive clearly that he must push on the trap instead of pulling it. It will be appreciated that it is possible to pull until the wings are broken, but the force necessary is nevertheless significant, and such as to make this a clear improper use of the device. For this reason, the movement of the component 68 which is intended to permit the extraction thereof from the cylindrical unit 8 should be understood as “one-way”, since, only by means of this movement is it possible to extract said component 68 without force after the “first blood” has been taken.

The invention, with the characteristics indicated in the following claims, provides a simple and efficient solution to the problem of potential contamination of the first blood taken, which blood can falsify the result of an examination, such as blood culture, of the blood taken from a patient.

1. A system for collecting blood taken from a blood vessel in a conventional phial, said system comprising a hollow retention unit which can receive said phial and contain it partially, said hollow retention unit receiving said blood and transferring the blood to said phial by means of a needle placed inside the retention unit, said retention unit having a first and a second portion and an inner cavity provided in said first and second portion, the retention unit having a first, open end in its first portion, said second portion of the retention unit having a second end in which said needle is present, inside said cavity there being inserted an element or trap which can receive a container for collection of a first blood taken in a test tube which in itself is known, said test tube being inserted in said element or trap and forming with the element or trap itself a single component which can be inserted into, and extracted from, said inner cavity of the retention unit, said element or trap having attachment means which can cooperate with attachment counter-means of the retention unit, said attachment means being able to be released in a one-way manner from said attachment counter-means after taking of the first blood which is collected in this container or test tube, the release of said attachment means and counter-means permitting the extraction of the element or trap from the cavity of the retention unit in order to permit the insertion therein of the phial, said collection means being lugs projecting from said element or trap which can cooperate with openings provided in said retention unit, said lugs projecting from the element or trap and being defined by wings which are hinged on a first part of the element or trap, characterised in that wherein said element or trap comprises a second part which extends from the first part, and defines a portion for containing the test tube, said second part comprising a cavity which can contain the test tube totally and is closed at the bottom at one end.

2. The system according to claim 1, wherein said second part of the element or trap defines a container portion comprising an inner cavity and having opposite lateral slots.

3. The system according to claim 2, wherein said second part of the element or trap has large openings in the vicinity of its closed end.

4. The system according to claim 1, wherein said second part of the element or trap is made of flexible material.

5. The system according to claim 1, wherein said second part of the element or trap contains the test tube by means of coupling by interference.

6. The system according to claim 1, wherein said release of the attachment means and counter-means is obtained when said element or trap is close to the second end of the cavity of said retention unit, this position being reached by moving said element or trap towards the second end of the cavity of the retention unit, from a position in which the cooperation of said attachment means and counter-means blocks said element or trap in said cavity.

7. The system according to claim 1, wherein the element or trap contains the container or test tube in a removable manner.

8. The system according to claim 1, wherein the element or trap contains the container or test tube in an inseparable manner.

9. The system according to claim 1, wherein said cylindrical unit has areas on which the wings or lugs slide during the movement of the component towards the second end of the cavity of the cylindrical unit.
